(19) United States
(12) Patent Application Publication
(10) Pub. No.: US 2017/0000747 A1
(43) Pub. Date: Jan. 5, 2017

(54) USE OF N-ACETYLCYSTEINE AMIDE IN THE TREATMENT OF ACETAMINOPHEN OVERDOSE

(71) Applicant: Glenn A. Goldstein, New York, NY (US)

(72) Inventor: Glenn A. Goldstein, New York, NY (US)

(21) Appl. No.: 15/174,089

(22) Filed: Jun. 6, 2016

Related U.S. Application Data
(60) Provisional application No. 62/171,710, filed on Jun. 5, 2015.

(51) Int. Cl. A61K 31/16; A61K 9/00; A61K 31/167

(52) U.S. Cl. CPC A61K 31/16; A61K 31/167; A61K 9/0053; A61K 9/0019

(57) ABSTRACT

This disclosure describes pharmaceutical compositions and methods of use for N-acetylcysteine amide for the prevention and treatment of acetaminophen overdose.

### CROSS-REFERENCE TO RELATED APPLICATIONS

This application is a non-provisional of U.S. patent application No. 62/171,710, filed Jun. 5, 2015, the contents of which is incorporated herein by reference in its entirety.

### BACKGROUND

Acetaminophen (N-acetyl-p-aminophenol, (APAP)) is one of the most widely used over-the-counter antipyretic analgesic medications. Despite being safe at therapeutic doses, an accidental or intentional overdose can result in severe hepatotoxicity; a leading cause of drug-induced liver failure in the U.S. Although a few different mechanisms have been proposed, a significant amount of evidence points to the potential involvement of oxidative stress in APAP toxicity. Depletion of glutathione (GSH) is believed to be a crucial initiating event in APAP toxicity and, therefore, N-acetylcysteine (NAC), a GSH precursor, is the only currently approved antidote for an APAP overdose. Unfortunately, fairly high doses and longer treatment times are required due to its poor bioavailability. In addition, oral and i.v. administration of NAC in a hospital setting are laborious and costly. Therefore, there is a dire need to develop therapeutic alternatives to effectively protect against APAP toxicity and to improve the outcome and prevent death.

### SUMMARY

The disclosure provides a pharmaceutical composition comprising 600-1200 mg of acetaminophen and 100-10,000 mg of N-acetylcysteine amide (NACA). In certain embodiments, the composition comprises 100-1000, 100-500 or 150-300 mg of NACA. In another embodiment, the composition further comprises a pharmaceutically acceptable salt or excipient.

In another embodiment, the pharmaceutical composition is a dosage form appropriate for oral administration. In certain aspects of this embodiment, the dosage form is selected from the group consisting of powders, granules, suspensions, slurries, solutions in water or non-aqueous media, sachets, capsules, gelcaps, lozenges, pills, dragees, gels, syrups and tablets.

In other embodiments, the pharmaceutical composition is a dosage form appropriate for intraperitoneal or intravenous administration.

The disclosure also provides a method of treating pain comprising in a subject in need thereof comprising administering to the subject any pharmaceutical composition described herein.

The disclosure also provides a method of treating N-acetyl-p-aminophenol (APAP) toxicity in a subject in need thereof comprising administering to the subject a therapeutically effective amount of NACA. In certain embodiments, therapeutically effective amount of NACA is between 100 and 10,000, 5000 and 10,000, or 7000 and 10,000 mg of NACA. In other embodiments, the therapeutically effective amount of NACA is between 50 and 200 or 80 and 150 mg/kg of NACA.

In another embodiment, the NACA is administered orally. In certain aspects of this embodiment, the dosage form is selected from the group consisting of powders, granules, suspensions, slurries, solutions in water or non-aqueous media, sachets, capsules, gelcaps, lozenges, pills, dragees, gels, syrups and tablets.

In other embodiments, the NACA is administered intraperitoneally or intravenously. In other embodiments, the subject is a mammal. In one aspect of this embodiment, the mammal is a human.

### BRIEF DESCRIPTION OF THE DRAWINGS

FIG. 1 shows bar graphs of GSH and cysteine (Cys) concentrations in plasma and liver of mice treated with NACA or NAC.

FIG. 2 shows bar graphs showing GSH concentrations in the liver, GSSG concentrations in plasma, MDA concentrations in plasma and GSH/GSSG plasma of mice treated with APAP, NAC and NACA as shown.

FIG. 3 shows bar graphs showing GR, glutamate dehydrogenase and ALT concentrations in the plasma of mice treated with APAP, NAC and NACA as shown.

FIG. 4 shows bar graphs showing GSH/GSSG and GR concentrations in the liver of mice treated with APAP, NAC and NACA as shown.

FIG. 5 shows bar graphs showing MDA concentrations in liver and GSH in mitochondria of mice treated with APAP, NAC and NACA as shown.

FIG. 6 shows bar graphs showing GSSG and GSH/GSSG concentrations in mitochondria of mice treated with APAP, NAC and NACA as shown.

FIG. 7 shows a bar graph showing GR concentrations in mitochondria of mice treated with APAP, NAC and NACA as shown.

FIG. 8 shows bar graphs showing GSH and Cys concentrations in kidney of mice treated with APAP, NAC and NACA as shown.

FIG. 9 shows bar graphs showing CK and BUN concentrations in serum of mice treated with APAP, NAC and NACA as shown.

FIG. 10 shows a line graph showing survival of mice adminstered APAP, NAC and NACA as shown.

### DETAILED DESCRIPTION

The present invention provides the use of N-acetylcysteine amide (NAC amide or NACA) or derivatives thereof, or a physiologically acceptable derivative, salt, or ester thereof, to treat or prevent acetaminophen (N-acetyl-p-aminophenol, (APAP)) toxicity. APAP toxicity results from an overdose of APAP in a subject. According to this invention NAC amide treatment can be prophylactic or therapeutic in treatment of APAP toxicity. NACA can be coadministered with APAP at a ratio where the dose of APAP necessary to cause toxicity in a subject is raised.

As used herein, a “subject” within the context of the present invention encompasses, without limitation, mammals, e.g., humans, domestic animals and livestock including cats, dogs, cattle and horses. A “subject in need thereof” is a subject having one or more manifestations of disorders, conditions, pathologies, and diseases as disclosed herein in which administration or introduction of NAC amide or its derivatives would be considered beneficial by those of ordinary skill in the art.

“Therapeutic treatment” or “therapeutic effect” means any improvement in the condition of a subject treated by the methods of the present invention, including obtaining a preventative or prophylactic effect, or any alleviation of the severity of signs or symptoms of APAP overdose. Symptoms of APAP overdose include nausea, vomiting, stomach pain, loss of appetite, paleness, tiredness, sweating, pain in the upper right side, dark colored urine, urinating less often, jaundice, blood in urine, fever, lightheadedness, fainting, troubled breathing, weakness, hunger, tremor, blurred vision, tachycardia, headache, somnolence, confusion and coma. In order to provide a therapeutic treatment, NACA must be administered in a therapeutic window after the APAP overdose. In certain embodiments, this window is between 0 and 96 hours.

“Prophylactic treatment” or “prophylactic effect” means prevention of APAP toxicity or raising the dosage of APAP necessary to cause symptoms associated with APAP toxicity in a subject. Typically, a toxic dose of APAP is more than 4000 mg in a four hour period. According to certain embodiments, by co-administering NACA with APAP, the toxic dose of APAP can be increased to between 5,000 and 100,000 mg of APAP. In certain embodiments, dosage forms of APAP contain NACA so that NACA must be combined with APAP when it is ingested.

NACA and derivatives thereof are provided, for example, in formula I:

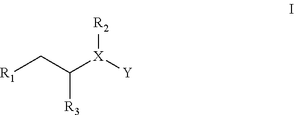

wherein: R₁ is OH, SH, or S—S—Z; X is C or N; Y is NH₂, OH, CH₃—C═O, or NH—CH₃; R.sub.2 is absent, H, or ═O R₃ is absent or

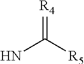

wherein: R₄ is NH or O; R₅ is CF₃, NH₂, or CH₃

and wherein: Z is

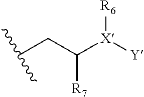

with the proviso that if R₁ is S—S—Z, X and X′ are the same, Y and Y′ are the same, R₂ and R₆ are the same, and R₃ and R₇ are the same.

The present invention also provides a NAC amide compound and NAC amide derivatives comprising the compounds disclosed herein. Other derivatives are disclosed in U.S. Pat. No. 8,354,449, incorporated by reference in its entirety.

In another aspect, a process for preparing an L- or D-isomer of NACA and derivatives thereof are provided, comprising adding a base to L- or D-cystine diamide dihydrochloride to produce a first mixture, and subsequently heating the first mixture under vacuum; adding a methanolic solution to the heated first mixture; acidifying the mixture with alcoholic hydrogen chloride to obtain a first residue; dissolving the first residue in a first solution comprising methanol saturated with ammonia; adding a second solution to the dissolved first residue to produce a second mixture; precipitating and washing the second mixture; filtering and drying the second mixture to obtain a second residue; mixing the second residue with liquid ammonia and an ethanolic solution of ammonium chloride to produce a third mixture; and filtering and drying the third mixture, thereby preparing the L- or D-isomer compound.

In some embodiments, the process further comprises dissolving the L- or D-isomer compound in ether; adding to the dissolved L- or D-isomer compound an ethereal solution of lithium aluminum hydride, ethyl acetate, and water to produce a fourth mixture; and filtering and drying the fourth mixture, thereby preparing the L- or D-isomer compound.

Another aspect of the invention provides a process for preparing an L- or D-isomer of the compounds disclosed herein, comprising mixing S-benzyl-L- or D-cysteine methyl ester hydrochloride or O-benzyl-L- or D-serine methyl ester hydrochloride with a base to produce a first mixture; adding ether to the first mixture; filtering and concentrating the first mixture; repeating steps (c) and (d), to obtain a first residue; adding ethyl acetate and a first solution to the first residue to produce a second mixture; filtering and drying the second mixture to produce a second residue; mixing the second residue with liquid ammonia, sodium metal, and an ethanolic solution of ammonium chloride to produce a third mixture; and filtering and drying the third mixture, thereby preparing the L- or D-isomer compound.

APAP Overdose

APAP overdose occurs when a dose of APAP is administered to a subject that causes symptoms of APAP toxicity. These symptoms of APAP toxicity include nausea, vomiting, stomach pain, loss of appetite, paleness, tiredness, sweating, pain in the upper right side, dark colored urine, urinating less often, jaundice, blood in urine, fever, lightheadedness, fainting, troubled breathing, weakness, hunger, tremor, blurred vision, tachycardia, headache, somnolence, confusion and coma. In certain embodiments, a dose of APAP that causes toxicity in an adult human subject is 4,000 mg or more. In other embodiments, the dose of APAP that causes toxicity in an adult human subject is 5,000, 5,500, 6,000, 6,500, 7,000, 7,500, 8,000, 8,500, 9,000, 9,500, 10,000 mg or more. In other embodiments, the dose of APAP that causes toxicity in a human child subject is 100 mg or more. In other embodiments, the dose of APAP that causes toxicity in a human child subject is 200, 300, 400, 500, 600, 700, 800, 900, 1000 mg or more. In other embodiments, a dose of APAP that causes toxicity in an adult human subject that has compromised liver function is 100 mg or more. In other embodiments, the dose of APAP that causes toxicity in an adult human subject that has compromised liver function is 200, 300, 400, 500, 600, 700, 800, 900, 1000 mg or more.

In certain embodiments, when a subject has received an APAP overdose in order to treat the overdose and symptoms associated therewith, NACA must be administered within a therapeutic window after the overdose. In certain embodiments, this therapeutic window is less than 24 hours from the onset of symptoms associated with APAP overdose or less than 24 hours from the administration of the overdose of APAP. In other embodiments, this therapeutic window is 1, 2, 3, 4, 5, 6, 12, 24, 36, 48, 60, 72, 84, 96, 120, 144, 168 or 200 hours. The length of the therapeutic window can depend on the amount of APAP administered in the overdose, the timing of the administration of APAP in the overdose, the size of the subject as well as the liver function of the subject.

In certain embodiments, when administering NACA to treat an APAP overdose, 100-100,000 mg of NACA are administered. In other embodiments, 1000-10,000, 2000-10,000, 3000-10,000, 4000-10,000, 5000-10,000, 6000-10,000, 7,000-10,000, 8000-10,000, 9000-10,000 mg are administered to an adult human subject. In other embodiments, 1000-5000, 2000-5000, 3000-5000, or 4000-5000 mg of APAP are administered to human child subject or a subject with compromised liver function. NACA can be administered according to any method known in the art. In certain embodiments, the NACA is administered orally, intraperitoneally or intravenously.

In other embodiments, NACA can be administered prior to or combined with an APAP overdose to reduce the severity of symptoms associated with an overdose and/or increase the amount of APAP that is needed to cause an overdose. In certain embodiments, this is accomplished by administering NACA at the same time or at approximately the same time as APAP. In certain embodiments, NACA is administered within 5 minutes of APAP administration. In other embodiments, NACA is administered within 10, 15, 30, 45 or 60 minutes of APAP administration. In some of these embodiments, NACA and APAP are administered in separate dosage forms.

In other embodiments, NACA and APAP are administered simultaneously. In certain embodiments, NACA and APAP are administered simultaneously and are in the same dosage form or pharmaceutical composition. When NACA and APAP are administered in the same dosage form or pharmaceutical composition they are administered in a ratio that reduces the severity of any potential APAP overdose and/or increases the amount of APAP necessary to cause an overdose and one or more of the symptoms associated therewith. In certain embodiments, a dosage form containing both APAP and NACA has a ratio of APAP to NACA of about 1:1. In other embodiments, the ratio of APAP to NACA is between 2:1 and 1:2; 10:1 and 1:1; 1:1 and 1:10; or 6:1 and 1:12.

According to certain embodiments, when NACA is administered with APAP to prevent the symptoms of overdose, APAP is administered at between 600 and 1200 mg and NACA is administered at between 100 and 10,000 mg. According to certain embodiments, these dosages are for a single dosage form. In certain circumstances, a subject may ingest an inappropriate amount of APAP in these dosage forms, that would ordinarily cause an APAP overdose. The presence of NACA reduces the chances that an overdose occurs by preventing the symptoms of overdose. In certain embodiments, the presence of NACA allows for a higher dose of APAP to be administered without risk of overdose. Thus APAP could be administered at doses of 1000-10,000, 2000-10,000, 3000-10,000, 4000-10,000, 5000-10,000, 6000-10,000, 7,000-10,000, 8000-10,000, 9000-10,000, 1000-5000, 2000-5000, 3000-5000, or 4000-5000 mg with doses of NACA of 100-10,000, 100-5000, 100-4000, 100-3000, 100-1000, 100-900, 100-800, 100-700, 100-600 or 100-500 mg.

Pharmaceutical Compositions

As used herein the term “pharmaceutical composition” refers to a preparation of one or more of the components described herein, or physiologically acceptable salts or prodrugs thereof, with other chemical components such as physiologically suitable carriers and excipients. The purpose of a pharmaceutical composition is to facilitate administration of a compound to an organism. The term “prodrug” refers a precursor compound that can hydrolyze, oxidize, or otherwise react under biological conditions (in vitro or in vivo) to provide the active compound. Examples of prodrugs include, but are not limited to, metabolites of NSAIDs that include biohydrolyzable moieties such as biohydrolyzable ainides, biohydrolyzable esters, biohydrolyzable carbarnates, biohydrolyzable carbonates, biohydrolyzable ureides, and biohydrolyzable phosphate analogues.

The term “excipient” refers to an inert or inactive substance added to a pharmaceutical composition to further facilitate administration of a compound. Non-limiting examples of excipients include calcium carbonate, calcium phosphate, various sugars and types of starch, cellulose derivatives, gelatin, vegetable oils and polyethylene glycols.

The pharmaceutical compositions of the present invention comprise NAC Amide or derviate thereof and may also include APAP. Pharmaceutical compositions that include NACA and APAP can be formulated into a single dosage form. In certain embodiments, this dosage form is an oral dosage form. This oral dosage form can be in the form of tablets, pills, dragees, capsules, liquids (aqueous or non-aqueous solutions), gels, syrups, slurries, gelcaps, lozenges, suspensions, and the like, for oral ingestion by a patient. In certain embodiments, one or more of the APAP or NACA are in a a slow release composition or have been formulated to affect release from the oral dosage form. In other embodiments, these dosage forms can be administered by any method known in the art including intravenously and intraperitoneally.

The pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

The term “administration” or any lingual variation thereof as used herein is meant any way of administration. The one or more of NAC Amide or derivative thereof and at least one additional drug may be administered in one therapeutic dosage form or in two separate therapeutic dosages such as in separate capsules, tablets or injections. In the case of the two separate therapeutic dosages, the administration may be such that the periods between the administrations vary or are determined by the practitioner. It is however preferred that the second drug is administered within the therapeutic response time of the first drug. The one or more of NAC Amide or derivative thereof and at least one additional drug which may be administered either at the same time, or separately, or sequentially, according to the invention, do not represent a mere aggregate of known agents, but a new combination with the valuable property that the effectiveness of the treatment is achieved at a much lower dosage of said at least one additional drug.

The pharmaceutical compositions of the present invention may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local.

Various delivery systems are known, e.g., encapsulation in liposomes, microparticles, microcapsules or capsules, that may be used to administer the compositions of the invention. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically to the cars, nose, eyes, or skin. The preferred mode of administration is left to the discretion of the practitioner, and will depend in part upon the site of the medical condition (such as the site of cancer) and the severity of thereof.

For example, for injection the composition of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the composition can be formulated readily by combining the active components with any pharmaceutically acceptable carriers known in the art. Such “carriers” may facilitate the manufacture of such as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Dyestuffs or pigments may be added to the tablets or dragee coatings for identification or to characterize different combinations of NACA and/or APAP doses. In addition, stabilizers may be added.

Pharmaceutical compositions for parenteral administration include aqueous solutions of the active preparation in a water-soluble form. Additionally, suspensions of the active preparation may be prepared as oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl, cellulose, sorbitol or dextran. Optionally, the suspension may also contain suitable stabilizers or agents, which increase the solubility of the compounds, to allow for the preparation of highly concentrated solutions.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

### EXAMPLES

### Example 1

### Comparative Evaluation of NAC and NACA in APAP-Induced Hepatotoxicity in C57BL/6 Mice

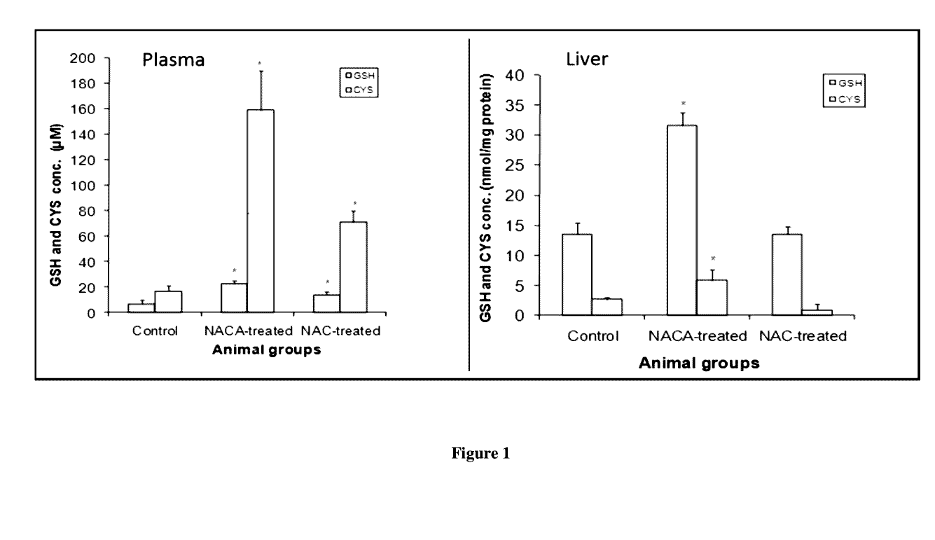

Wistar rats were divided into two groups (three animals per group) and administered APAP at 400 mg/kg followed by either NACA or NAC, using i.p. injections of 106 mg/kg of each compound. After 30 minutes, the rats were anesthetized and their blood collected via cardiopuncture. The animals were then euthanized and their livers harvested. Plasma and liver tissue samples were prepared for analysis of GSH and CYS levels. This analysis showed that NACA was significantly more effective than NAC at providing thiols in plasma and liver tissue (FIG. 1).

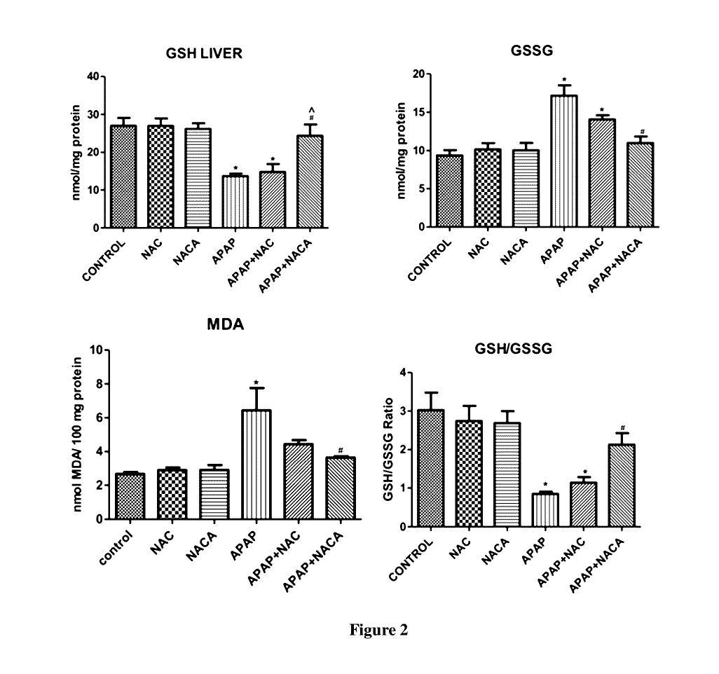

Studies were then performed to evaluate the protective effects of NACA in APAP-induced hepatotoxicity using male C57BL/6 mice that were intraperitoneally injected with 400 mg/kg of APAP after 15 hours of fasting. The therapeutic potentials of NAC and NACA were compared by administering NAC/NACA (106 mg/kg) after 1.5 hours of APAP administration and sacrificing the mice 4 hours after the APAP administration. The GSH/GSSG ratio, a crucial antioxidant parameter, and malondialdehyde (MDA) levels were measured in liver tissue. The GSH/GSSG ratio in NACA treated rats had increased significantly when compared with NAC and MDA levels remained similar to controls (FIG. 2).

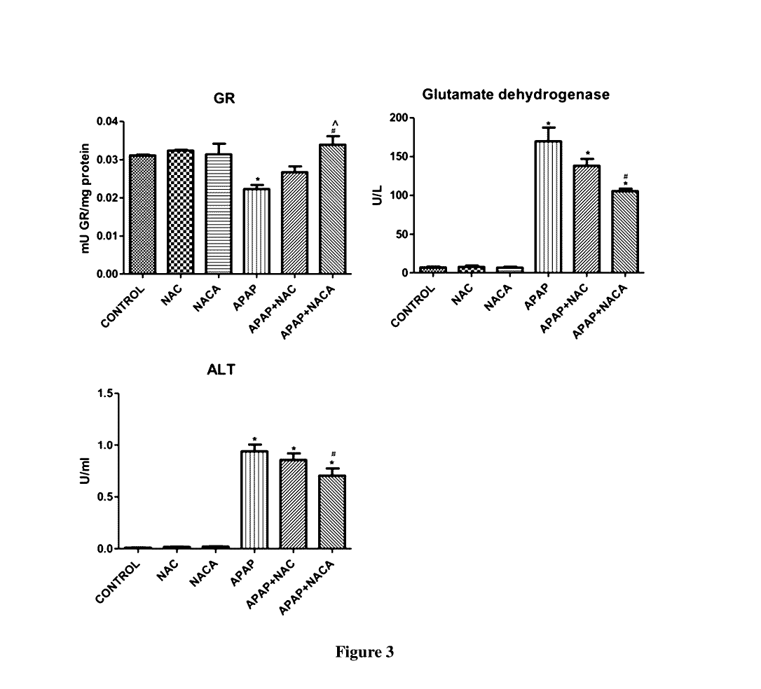

Alanine aminotransferase (ALT) and glutamate dehydrogenase levels were also measured in serum (FIG. 3). These are markers of livery injury. APAP causes liver injury that is reversed by NACA.

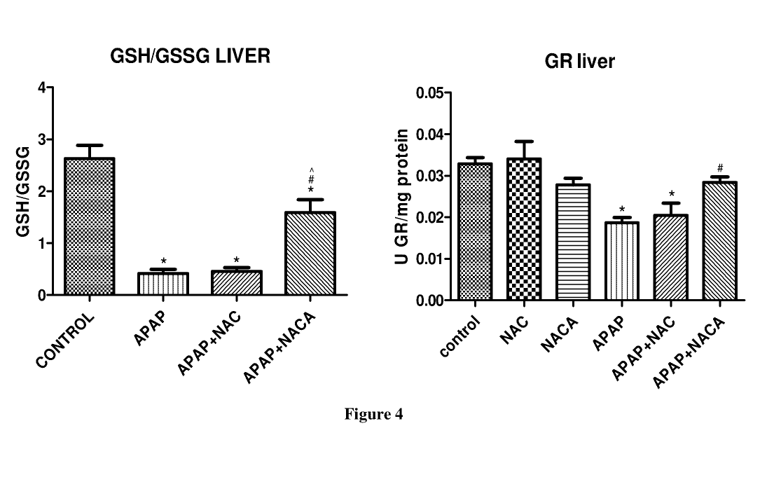

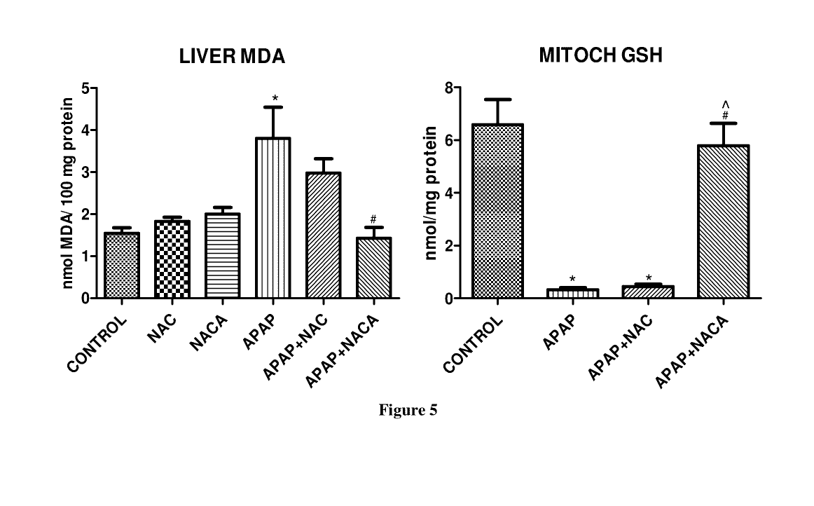

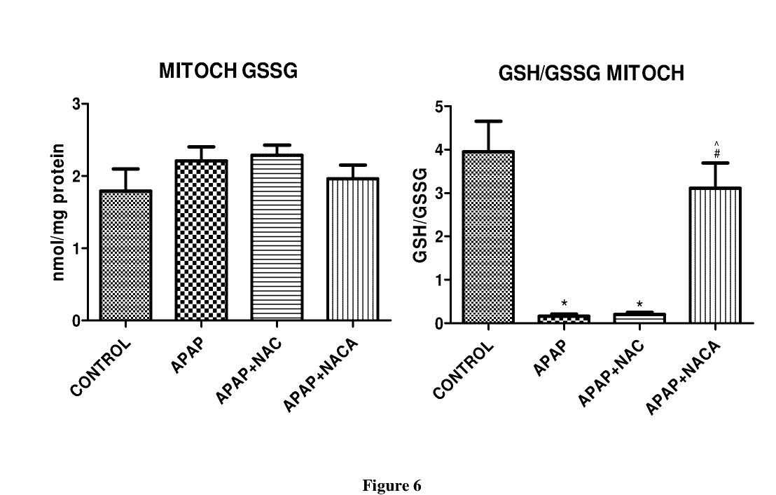

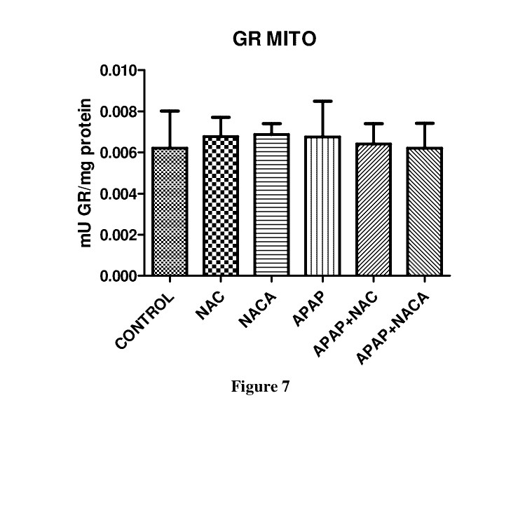

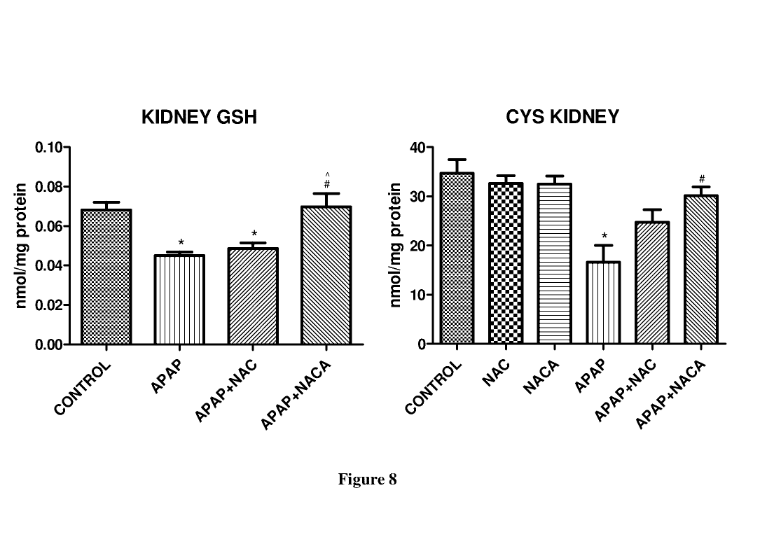

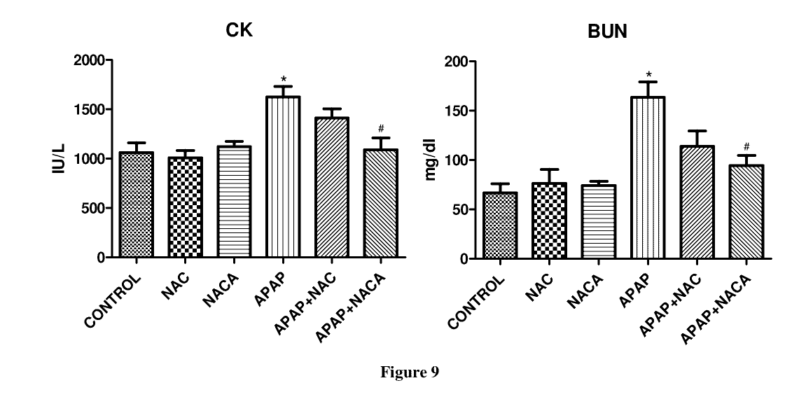

The administration of APAP increases oxidative stress in a number of tissues that is significantly reduced by administration of NACA. FIGS. 4-8 show increases in GSH/GSSG, MDA, Cys and glutathione reductase (GR) in liver, mitochondria, and kidney when NACA is administered. FIG. 9 shows that APAP increases serum creatine kinase (CK) and blood urea nitrogen (BUN) while administration of NACA significantly reduces them.

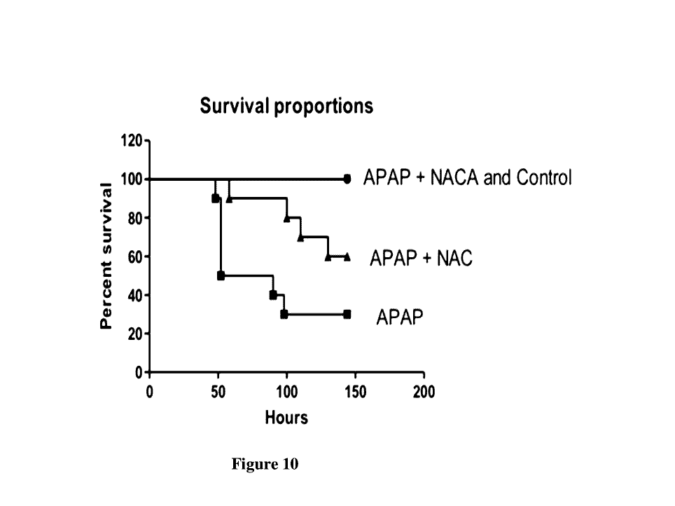

Studies were then performed where C57 BL/6 mice received APAP overdose of 500 mg/kg. NACA or NAC was administered (106 mg/kg) 1.5 hours post APAP and at every 12 hours thereafter until 72 hours. The APAP group had 6 mice and the NAC and NACA gruos contained 4. Mice treated with NACA/APAP survived for 150 hours, while mice treated with APAP or NAC/APAP had 60% and 30% survival respectively (FIG. 10).

### Example 2

### Comparison of the Rescue Ability of NACA with that of NAC by Studying Survival of Mice with APAP-Induced Acute Liver Failure

C57BL/6 mice (Charles River laboratories), at an average age of 7 to 9 weeks, will be housed in an environmentally controlled room with a 12 hours light/dark cycle and fed with standard food and water ad libitum. The animals will be acclimatized for a minimum of 1 week before the experiment. All experimental procedures will be conducted under an animal protocol approved by the Institutional Animal Care and Use Committee at Missouri University of Science and Technology.

Mice will be fasted overnight prior to APAP treatment. APAP will be dissolved in warm saline (15 mg/ml). A NAC/NACA solution will be made fresh in PBS at 20 mg/ml. For the first set of studies, C57BL/6 mice (n=10) will be given 200-800 mg/kg APAP i.p. and observed for survival for 48 hours. A dose response curve will be generated to calculate the lethal dose.

To compare the ability of NACA and NAC to rescue from APAP toxicity, the animals will be randomly divided into six groups (n=10) (Table 2): a) Control; b) APAP only; c) NACA only; d) NAC only; e) APAP+NAC; and f) APAP+NACA. The mice will be administered a lethal dose of APAP followed by an i.p. injection of NAC/NACA (106 mg/kg) 1 hour after the APAP dose. A sub therapeutic dose of NAC is selected for these studies to determine if NACA would be better than NAC at a lower dose. The animals will be monitored for 72 hours to compare the rescue ability of NAC and NACA against APAP-induced toxicity

TABLE 2
Treatment groups to be studied

| Groups (n = 10) | Injection (i.p.) |
|---|---|
| Control | Vehicle for APAP + Vehicle for NAC/NACA |
| NAC only | Vehicle for APAP + NAC Injection (106 mg/kg) |
| NACA only | Vehicle for APAP + NACA Injection (106 mg/kg) |
| APAP | APAP injection (lethal dose) + Vehicle for |
| | NAC/NACA. |
| APAP + NAC | APAP injection (lethal dose) + NAC Injection |
| | (106 mg/kg after 1 hour) |
| APAP + NACA | APAP injection (lethal dose) + NACA Injection |
| | (106 mg/kg after 1 hour) |

### Example 3

### Comparison of the Therapeutic Potential of NACA with that of NAC in APAP-Induced Organ Damage

C57BL/6 mice will be divided them into six groups (n=24): a) untreated control; b) NAC only (106 mg/kg); c) NACA only (106 mg/kg); d) APAP-only (400 mg/kg); e) APAP+NACA; and f) APAP+NAC. Acute liver failure will be induced by i.p. injections of APAP (a sub lethal dose: approximately 400 mg/kg) after overnight food deprivation. 106 mg/kg NACA/NAC will be administered 1 hour after a 400 mg/kg APAP injection followed by three more i.p. injections of NAC/NACA every 12 hours for 36 hours. Six mice each will be sacrificed at 4, 16, 28, and 40 hours after APAP administration to assess organ damage. Blood, liver, and kidney tissues will be harvested. Whole blood samples will be allowed to clot and then centrifuged at 9000 g for 30 minutes. Serum will be collected to measure lactate dehydrogenase (LDH), glutamate dehydrogenase (GDH), aspartate transaminase (AST), alanine transaminase (ALT), blood urea nitrogen (BUN), and creatinine levels Immediately after collecting the blood, the organs will be harvested and rinsed in saline. A small section from a liver as well as a kidney will be fixed in 4%-10% phosphate buffered formalin to be used for H&E staining. The remaining organs will be frozen in liquid nitrogen and stored at −80° C. for further analysis. In addition, to investigate the role of oxidative stress, various stress parameters (such as intracellular GSH, GSSG, protein carbonyls, and MDA levels) and activities of antioxidant enzymes (such as glutathione peroxidase, GR, catalase, and SOD activity) will be measured. Since mitochondrial dysfunction is implicated in APAP-induced toxicity, the effect of NAC/NACA in preserving mitochondrial bioenergetics will be assessed by measuring mitochondrial respiration. To further support the mitochondrial respiration results, the effect of NACA treatment on activities of individual enzyme complexes that are involved in mitochondrial energy production will also be assessed. In addition the GSH/GSSG ratio will be determined in the mitochondria. Liver oxidative injury induced by acute treatment with APAP is reported to be accompanied by neutrophil infiltration. To compare the hepatoprotective actions of NAC/NACA in terms of their ability to modulate neutrophil-related oxidative stress reactions, MPO activity will be measured. Furthermore, the extent of necrosis will be correlated with DNA fragmentation (TUNEL assay) and peroxynitrite formation (nitrotyrosine protein adduct formation). In addition, the phosphorylation of JNK will be studied using Western blotting.

### Example 4

### Comparison if the Therapeutic Potential of NAC/NACA Against APAP-Induced Toxicity After Delayed Application

C57BL/6 mice (7 weeks old) will used to compare the therapeutic potential of NACA and NAC against APAP toxicity after delayed administration. The animals will be randomly divided into six major groups, based on the time of NAC/NACA injection administration. Each group (n=36) will receive six different treatments as exemplified in Table 3. The mice will be administered a sub lethal dose of APAP followed by only one i.p. injection of NAC/NACA (106 mg/kg). The major groups will differ only in the time of administration of NAC/NACA after the APAP dose (i.e.; 1, 4, 8, 12, 16, and 20 hours after the APAP dose). The animals will be sacrificed 24 hours after the APAP dose by cervical dislocation. Blood will be drawn from the vena cava into heparinized syringes and centrifuged.

Immediately after collecting the blood, the organs will be excised and rinsed in saline. A small section from each liver and kidney will be placed in phosphate-buffered formalin to be used in immunohistochemical analysis. A portion of the remaining liver will be homogenized for isolation of the mitochondria. The remaining tissues will be frozen in liquid nitrogen and stored at −80° C. for later analysis of oxidative stress parameters, as detailed in SA 2.

TABLE 3
Treatment sub groups to be studied (Major groups will differ
in the time of administration of NAC/NACA after APAP dose)

| Sub Groups (n = 6) | Injection (i.p.) |
|---|---|
| Control | Vehicle for APAP + Vehicle for NAC/NACA |
| NAC only | Vehicle for APAP + NAC Injection (106 mg/kg) |
| NACA only | Vehicle for APAP + NACA injection (106 mg/kg) |
| APAP | APAP injection (lethal dose) + Vehicle for |
| | NAC/NACA |
| APAP + NAC | APAP injection (lethal dose) + NAC Injection |
| | (106 mg/kg after 1 hour) |
| APAP + NACA | APAP injection (lethal dose) + NACA Injection |
| | (106 mg/kg after 1 hour) |

Experimental Methods

Research Animals: For these experiments, 6-week old C57BL/6 mice will be purchased from the Jackson Laboratory (Bar Harbor, Maine). The animals will be housed in groups of five and kept in controlled temperature (22±1° C.) and humidity (60-70%), under a 12-hour light-dark cycle (lights on 07:00 AM).

Food and water will be available ad libitum; however, mice will be fasted 16 hours prior to treatment with APAP. Mice will be adapted to the laboratory for a period of 1 week prior to experimental procedures.

Animals will be euthanized at the end of the experimental period by cervical dislocation. All animal procedures will be conducted under an animal protocol approved by the Institutional Animal Care and Use Committee of the Missouri University of Science and Technology.

Measurement of Intracellular GSH and GSSG Levels: The levels of GSH and GSSG will be measured according to our previously published article.⁵¹

Measurement of Malondialdehyde (MDA) Levels: Lipid peroxidation is estimated by measuring MDA levels. MDA levels will be measured in whole cell homogenates using butylated hydroxytoluene (BHT) and trichloroacetic acid (TCA) and analyzed by HPLC with flurescence detection. The levels of MDA will be quantified relative to a standard curve.

Measurement of Protein Carbonyl Levels: The protein carbonyl level will be measured, as previously published.⁵³

Antioxidant Enzyme Activities: Activity of SOD will be determined as described by Gardener et al.⁵⁴ Catalase activity will be determined spectrophotometrically in cell homogenates and expressed in units/mg of protein, as described by Aebi et al.⁵⁵ Glutathione peroxidase and reductase activity will be determined using a test kit (OxisResearch).

Determination of serum ALT and AST activity: Following euthanization, blood samples will be collected. Serum will be separated and stored at −80° C. until further analysis. ALT and AST levels will be measured using commercially available kits according to the manufacturer's recommendations (Labtest, Brazil). The results will be read at 505 nm, and the final results will be calculated on the basis of a calibration curve.

Determination of serum BUN and creatinine levels: Following euthanization, blood samples will be collected. Serum will be separated and stored at −80° C. until further analysis. BUN and creatinine levels will be measured using commercially available kits according to the manufacturer's recommendations (Pointe Scientific, U.S.A.).

Mitochondrial isolation: The liver will be isolated, blotted, weighed and placed in mitochondrial Isolation Buffer A (215 mM mannitol, 75 mM sucrose, 0.1% BSA, 20 mM HEPES, and 1 mM EGTA; pH 7.2). The liver will be minced and homogenized in a Dounce homogenizer on ice. Following homogenization, the liver will be centrifuged at 1300 rcf for 3 minutes. The resultant pellet will be discarded and the supernatant will be centrifuged at 13,000 rcf for 10 minutes. The pellet containing the mitochondria will be resuspended in mitochondrial isolation buffer and samples will be stored at −80° C. until analysis.

Measurement of mitochondrial function: Measurement of mitochondrial functions will be done as reported by Patel et al.⁵⁶

Neutrophil myeloperoxidase assay: Neutrophil recruitment will be measured by means of tissue myeloperoxidase (MPO) activity as described by Souza et al.⁵⁷ with minor modifications. The liver tissues will be removed, homogenized at 5% (w/v) in EDTA/NaCl buffer (pH 4.7) and centrifuged at 10,000 rpm for 15 minutes at 4° C. The pellet will be resuspended in 0.5% hexadecyltrimethyl ammonium bromide buffer (pH 5.4) and the samples will be frozen. Upon thawing, the samples will be re-centrifuged (10,000 rpm, 15 minutes, 4° C.) and 25 μl of the supernatant will be used for MPO assay. The enzymatic reaction will be assessed with 1.6 mM tetramethylbenzidine, 80 mM NaPO4, and 0.3 mM hydrogen peroxide. The absorbance will be measured at 595 nm, and the results will be expressed as OD per milligram of tissue.

Histology and immunohistochemistry: Formalin-fixed tissue samples will be embedded in paraffin and 5 μm sections will be cut. Replicate sections will be stained with hematoxylin and eosin (H&E) for evaluation of necrosis. For the terminal deoxynucleotidyl transferase-mediated dUTP nick-end labeling (TUNEL) assay, sections of liver will be stained with the In Situ Cell Death Detection Kit, AP (Roche Diagnostics, Indianapolis, Ind.) as described in the manufacturer's instructions. Nitrotyrosine protein adducts will be detected with standard immunohistochemical methods using an anti-nitrotyrosine antibody (Molecular Probes, Eugene, Oreg.).

Expression and Modifications of Proteins: Western blotting for JNK will be performed using tissue homogenates with anti-JNK and anti-phospho-JNK antibodies from Cell Signaling (Danvers, Mass.). Briefly, tissue homogenates will be resolved by SDS-PAGE, followed by immunoblotting on a nitrocellulose membrane (Bio-Rad, Hercules, Calif.). Following standard procedures, the membrane will be probed with respective antibodies and the immunoreactivity will be detected by a chemiluminescent method (Pierce, Rockford, Ill.). GAPDH will be used as an internal control to ensure equal loading of protein.

Statistical Analysis: Group comparisons will be performed using the one-way analysis of variance (ANOVA) test and TUKEY'S post hoc test. Statistical analyses will be made using GraphPad Prism 5.01 (GraphPad Software Inc., La Jolla, Calif.). Statistical significance will be set at p<0.05.

### REFERENCES

(1) Bertolini, A.; Ferrari, A.; Ottani, A.; Guerzoni, S.; Tacchi, R.; Leone, S.: Paracetamol: new vistas of an old drug. CNS drug reviews 2006, 12, 250-75.

(2) Myers, S. H.; LaPorte, D. M.: Acetaminophen: safe use and associated risks. The Journal of hand surgery 2009, 34, 1137-9.

(3) Smith, H. S.: Potential analgesic mechanisms of acetaminophen. Pain physician 2009, 12, 269-80.

(4) Jaeschke, H.; Williams, C. D.; McGill, M. R.; Farhood, A.: Herbal extracts as hepatoprotectants against acetaminophen hepatotoxicity. World journal of gastroenterology: WJG 2010, 16, 2448-50.

(5) Nourjah, P.; Ahmad, S. R.; Karwoski, C.; Willy, M.: Estimates of acetaminophen (Paracetomal)-associated overdoses in the United States. Pharmacoepidemiology and drug safety 2006, 15, 398-405.

(6) McGill, M. R.; Li, F.; Sharpe, M. R.; Williams, C. D.; Curry, S. C.; Ma, X.; Jaeschke, H.: Circulating acylcarnitines as biomarkers of mitochondrial dysfunction after acetaminophen overdose in mice and humans. Archives of toxicology 2014, 88, 391-401.

(7) Varney, S. M.; Buchanan, J. A.; Kokko, J.; Heard, K.: Acetylcysteine for acetaminophen overdose in patients who weigh >100 kg. American journal of therapeutics 2014, 21, 159-63.

(8) Polson, J.; Lee, W. M.: AASLD position paper: the management of acute liver failure. Hepatology 2005, 41, 1179-97.

(9) Larson, A. M.; Polson, J.; Fontana, R. J.; Davern, T. J.; Lalani, E.; Hynan, L. S.; Reisch, J. S.; Schiodt, F. V.; Ostapowicz, G.; Shakil, A. O.; Lee, W. M.: Acetaminophen-induced acute liver failure: results of a United States multicenter, prospective study. Hepatology 2005, 42, 1364-72.

(10) Nelson, S. D.: Molecular mechanisms of the hepatotoxicity caused by acetaminophen. Seminars in liver disease 1990, 10, 267-78.

(11) McGill, M. R.; Sharpe, M. R.; Williams, C. D.; Taha, M.; Curry, S. C.; Jaeschke, H.: The mechanism underlying acetaminophen-induced hepatotoxicity in humans and mice involves mitochondrial damage and nuclear DNA fragmentation. The Journal of clinical investigation 2012, 122, 1574-83.

(12) Dahlin, D. C.; Nelson, S. D.: Synthesis, decomposition kinetics, and preliminary toxicological studies of pure N-acetyl-p-benzoquinone imine, a proposed toxic metabolite of acetaminophen. Journal of medicinal chemistry 1982, 25, 885-6.

(13) Chen, C.; Hennig, G. E.; Manautou, J. E.: Hepatobiliary excretion of acetaminophen glutathione conjugate and its derivatives in transport-deficient (TR-) hyperbilirubinemic rats. Drug metabolism and disposition: the biological fate of chemicals 2003, 31, 798-804.

(14) Mitchell, J. R.; Jollow, D. J.; Potter, W. Z.; Davis, D. C.; Gillette, J. R.; Brodie, B. B.: Acetaminopheninduced hepatic necrosis. I. Role of drug metabolism. The Journal of pharmacology and experimental therapeutics 1973, 187, 185-94.

(15) McGill, M. R.; Lebofsky, M.; Norris, H. R.; Slawson, M. H.; Bajt, M. L.; Xie, Y.; Williams, C. D.; Wilkins, D. G.; Rollins, D. E.; Jaeschke, H.: Plasma and liver acetaminophen-protein adduct levels in mice after acetaminophen treatment: dose-response, mechanisms, and clinical implications. Toxicology and applied pharmacology 2013, 269, 240-9.

(16) Coles, B.; Wilson, I.; Wardman, P.; Hinson, J. A.; Nelson, S. D.; Ketterer, B.: The spontaneous and enzymatic reaction of N-acetyl-p-benzoquinonimine with glutathione: a stopped-flow kinetic study. Archives of biochemistry and biophysics 1988, 264, 253-60.

(17) Potter, W. Z.; Davis, D. C.; Mitchell, J. R.; Jollow, D. J.; Gillette, J. R.; Brodie, B. B.: Acetaminopheninduced hepatic necrosis. 3. Cytochrome P-450-mediated covalent binding in vitro. The Journal of pharmacology and experimental therapeutics 1973, 187, 203-10.

(18) Jaeschke, H.; Williams, C. D.; McGill, M. R.; Xie, Y.; Ramachandran, A.: Models of drug-induced liver injury for evaluation of phytotherapeutics and other natural products. Food and chemical toxicology: an international journal published for the British Industrial Biological Research Association 2013, 55, 279-89.

(19) Jaeschke, H.; Bajt, M. L.: Intracellular signaling mechanisms of acetaminophen-induced liver cell death. Toxicological sciences: an official journal of the Society of Toxicology 2006, 89, 31-41.

(20) Gunawan, B. K.; Liu, Z. X.; Han, D.; Hanawa, N.; Gaarde, W. A.; Kaplowitz, N.: c-Jun N-terminal kinase plays a major role in murine acetaminophen hepatotoxicity. Gastroenterology 2006, 131, 165-78.

(21) Saito, C.; Lemasters, J. J.; Jaeschke, H.: c-Jun N-terminal kinase modulates oxidant stress and peroxynitrite formation independent of inducible nitric oxide synthase in acetaminophen hepatotoxicity. Toxicology and applied pharmacology 2010, 246, 8-17.

(22) Nakagawa, H.; Maeda, S.; Hikiba, Y.; Ohmae, T.; Shibata, W.; Yanai, A.; Sakamoto, K.; Ogura, K.; Noguchi, T.; Karin, M.; Ichijo, H.; Omata, M.: Deletion of apoptosis signal-regulating kinase 1 attenuates acetaminophen-induced liver injury by inhibiting c-Jun N-terminal kinase activation. Gastroenterology 2008, 135, 1311-21.

(23) Sharma, M.; Gadang, V.; Jaeschke, A.: Critical role for mixed-lineage kinase 3 in acetaminopheninduced hepatotoxicity. Molecular pharmacology 2012, 82, 1001-7.

(24) Elsby, R.; Kitteringham, N. R.; Goldring, C. E.; Lovatt, C. A.; Chamberlain, M.; Henderson, C. J.; Wolf, C. R.; Park, B. K.: Increased constitutive c-Jun N-terminal kinase signaling in mice lacking glutathione S-transferase Pi. The Journal of biological chemistry 2003, 278, 22243-9.

(25) Hanawa, N.; Shinohara, M.; Saberi, B.; Gaarde, W. A.; Han, D.; Kaplowitz, N.: Role of JNK translocation to mitochondria leading to inhibition of mitochondria bioenergetics in acetaminophen-induced liver injury. The Journal of biological chemistry 2008, 283, 13565-77.

(26) Win, S.; Than, T. A.; Han, D.; Petrovic, L. M.; Kaplowitz, N.: c-Jun N-terminal kinase (JNK)-dependent acute liver injury from acetaminophen or tumor necrosis factor (TNF) requires mitochondrial Sab protein expression in mice. The Journal of biological chemistry 2011, 286, 35071-8.

(27) Kon, K.; Kim, J. S.; Jaeschke, H.; Lemasters, J. J.: Mitochondrial permeability transition in acetaminophen-induced necrosis and apoptosis of cultured mouse hepatocytes. Hepatology 2004, 40, 1170-9.

(28) Bajt, M. L.; Farhood, A.; Lemasters, J. J.; Jaeschke, H.: Mitochondrial bax translocation accelerates DNA fragmentation and cell necrosis in a murine model of acetaminophen hepatotoxicity. The Journal of pharmacology and experimental therapeutics 2008, 324, 8-14.

(29) Bajt, M. L.; Cover, C.; Lemasters, J. J.; Jaeschke, H.: Nuclear translocation of endonuclease G and apoptosis-inducing factor during acetaminophen-induced liver cell injury. Toxicological sciences: an official journal of the Society of Toxicology 2006, 94, 217-25.

(30) Bajt, M. L.; Ramachandran, A.; Yan, H. M.; Lebofsky, M.; Farhood, A.; Lemasters, J. J.; Jaeschke, H.: Apoptosis-inducing factor modulates mitochondrial oxidant stress in acetaminophen hepatotoxicity. Toxicological sciences: an official journal of the Society of Toxicology 2011, 122, 598-605.

(31) Latchoumycandane, C.; Goh, C. W.; Ong, M. M.; Boelsterli, U. A.: Mitochondrial protection by the JNK inhibitor leflunomide rescues mice from acetaminophen-induced liver injury. Hepatology 2007, 45, 412-21.

(32) Antoine, D. J.; Jenkins, R. E.; Dear, J. W.; Williams, D. P.; McGill, M. R.; Sharpe, M. R.; Craig, D. G.; Simpson, K. J.; Jaeschke, H.; Park, B. K.: Molecular forms of HMGB1 and keratin-18 as mechanistic biomarkers for mode of cell death and prognosis during clinical acetaminophen hepatotoxicity. Journal of hepatology 2012, 56, 1070-9.

(33) Wendel, A.; Feuerstein, S.; Konz, K. H.: Acute paracetamol intoxication of starved mice leads to lipid peroxidation in vivo. Biochemical pharmacology 1979, 28, 2051-5.

(34) Squadrito, G. L.; Pryor, W. A.: Oxidative chemistry of nitric oxide: the roles of superoxide, peroxynitrite, and carbon dioxide. Free radical biology & medicine 1998, 25, 392-403.

(35) Beckman, J. S.: Oxidative damage and tyrosine nitration from peroxynitrite. Chemical research in toxicology 1996, 9, 836-44.

(36) Knight, T. R.; Ho, Y. S.; Farhood, A.; Jaeschke, H.: Peroxynitrite is a critical mediator of acetaminophen hepatotoxicity in murine livers: protection by glutathione. The Journal of pharmacology and experimental therapeutics 2002, 303, 468-75.

(37) Saito, C.; Zwingmann, C.; Jaeschke, H.: Novel mechanisms of protection against acetaminophen hepatotoxicity in mice by glutathione and N-acetylcysteine. Hepatology 2010, 51, 246-54.

(38) Grinberg, L.; Fibach, E.; Amer, J.; Atlas, D.: N-acetylcysteine amide, a novel cell-permeating thiol, restores cellular glutathione and protects human red blood cells from oxidative stress. Free radical biology & medicine 2005, 38, 136-45.

(39) Atlas D, M. E., Ofen D, inventors; Yissum M, assignee.: Brain targeted low molecular weight hydrophobic antioxidant compounds. U.S. patent U.S. Pat. No. 5,874,468 A.1999 Feb. 23.

(40) Cotgreave, I. A.: N-acetylcysteine: pharmacological considerations and experimental and clinical applications. Adv Pharmacol 1997, 38, 205-27.

(41) Tan, H. H.; Chang, C. Y.; Martin, P.: Acetaminophen hepatotoxicity: current management. The Mount Sinai journal of medicine, New York 2009, 76, 75-83.

(42) Marzullo, L.: An update of N-acetylcysteine treatment for acute acetaminophen toxicity in children. Current opinion in pediatrics 2005, 17, 239-45.

(43) Sandilands, E. A.; Bateman, D. N.: Adverse reactions associated with acetylcysteine. Clin Toxicol (Phila) 2009, 47, 81-8.

(44) Yarema, M. C.; Johnson, D. W.; Berlin, R. J.; Sivilotti, M. L.; Nettel-Aguirre, A.; Brant, R. F.; Spyker, D. A.; Bailey, B.; Chalut, D.; Lee, J. S.; Plint, A. C.; Purssell, R. A.; Rutledge, T.; Seviour, C. A.; Stiell, I. G.; Thompson, M.; Tyberg, J.; Dart, R. C.; Rumack, B. H.: Comparison of the 20-hour intravenous and 72-hour oral acetylcysteine protocols for the treatment of acute acetaminophen poisoning. Annals of emergency medicine 2009, 54, 606-14.

(45) Ates, B.; Abraham, L.; Ercal, N.: Antioxidant and free radical scavenging properties of Nacetylcysteine amide (NACA) and comparison with N-acetylcysteine (NAC). Free radical research 2008, 42, 372-7.

(46) Offen, D.; Gilgun-Sherki, Y.; Barhum, Y.; Benhar, M.; Grinberg, L.; Reich, R.; Melamed, E.; Atlas, D.: A low molecular weight copper chelator crosses the blood-brain barrier and attenuates experimental autoimmune encephalomyelitis. Journal of neurochemistry 2004, 89, 1241-51.

(47) Bahat-Stroomza, M.; Gilgun-Sherki, Y.; Offen, D.; Panet, H.; Saada, A.; Krool-Galron, N.; Barzilai, A.; Atlas, D.; Melamed, E.: A novel thiol antioxidant that crosses the blood brain barrier protects dopaminergic neurons in experimental models of Parkinson's disease. The European journal of neuroscience 2005, 21, 637-46.

(48) Gilgun-Sherki, Y.; Barhum, Y.; Atlas, D.; Melamed, E.; Offen, D.: Analysis of gene expression in MOG-induced experimental autoimmune encephalomyelitis after treatment with a novel brain penetrating antioxidant. Journal of molecular neuroscience: MN 2005, 27, 125-35.

(49) Penugonda, S.; Mare, S.; Goldstein, G.; Banks, W. A.; Ercal, N.: Effects of N-acetylcysteine amide (NACA), a novel thiol antioxidant against glutamate-induced cytotoxicity in neuronal cell line PC12. Brain research 2005, 1056, 132-8.

(50) Tobwala, S.; Fan, W.; Stoeger, T.; Ercal, N.: N-acetylcysteine amide, a thiol antioxidant, prevents bleomycin-induced toxicity in human alveolar basal epithelial cells (A549). Free radical research 2013, 47, 740-9.

(51) Winters, R. A.; Zukowski, J.; Ercal, N.; Matthews, R. H.; Spitz, D. R.: Analysis of glutathione, glutathione disulfide, cysteine, homocysteine, and other biological thiols by high-performance liquid chromatography following derivatization by n-(1-pyrenyl)maleimide. Analytical biochemistry 1995, 227, 14-21.

(52) Price, T. 0.; Ercal, N.; Nakaoke, R.; Banks, W. A.: HIV-1 viral proteins gp120 and Tat induce oxidative stress in brain endothelial cells. Brain research 2005, 1045, 57-63.

(53) Banerjee, A.; Zhang, X.; Manda, K. R.; Banks, W. A.; Ercal, N.: HIV proteins (gp120 and Tat) and methamphetamine in oxidative stress-induced damage in the brain: potential role of the thiol antioxidant N-acetylcysteine amide. Free radical biology & medicine 2010, 48, 1388-98.

(54) Gardner, P. R.; Raineri, I.; Epstein, L. B.; White, C. W.: Superoxide radical and iron modulate aconitase activity in mammalian cells. The Journal of biological chemistry 1995, 270, 13399-405.

(55) Aebi, H.: Catalase in vitro. Methods in enzymology 1984, 105, 121-6.

(56) Patel, S. P.; Sullivan, P. G.; Pandya, J. D.; Goldstein, G. A.; VanRooyen, J. L.; Yonutas, H. M.; Eldahan, K. C.; Morehouse, J.; Magnuson, D. S.; Rabchevsky, A. G.: N-acetylcysteine amide preserves mitochondrial bioenergetics and improves functional recovery following spinal trauma. Experimental neurology 2014, 257, 95-105.

(57) Souza, D. G.; Cassali, G. D.; Poole, S.; Teixeira, M. M.: Effects of inhibition of PDE4 and TNF-alpha on local and remote injuries following ischaemia and reperfusion injury. British journal of pharmacology 2001, 134, 985-94.

What is claimed is:

1. A pharmaceutical composition comprising 600-1200 mg of acetaminophen and 100-10,000 mg of N-acetylcysteine amide (NACA).

2. The pharmaceutical composition of claim 1, wherein the pharmaceutical composition comprises 100-1000 mg of NACA.

3. The pharmaceutical composition of claim 1, wherein the pharmaceutical composition comprises 100-500 mg of NACA.

4. The pharmaceutical composition of claim 1, wherein the pharmaceutical composition comprises 150-300 mg of NACA.

5. The pharmaceutical composition of claim 1, wherein the pharmaceutical composition further comprises a pharmaceutically acceptable salt or excipient.

6. The pharmaceutical composition of claim 1, wherein the pharmaceutical composition is a dosage form appropriate for oral administration.

7. The pharmaceutical composition of claim 4, wherein the dosage form is selected from the group consisting of powders, granules, suspensions, slurries, solutions in water or non-aqueous media, sachets, capsules, gelcaps, lozenges, pills, dragees, gels, syrups and tablets.

8. The pharmaceutical composition of claim 1, wherein the pharmaceutical composition is a dosage form appropriate for intraperitoneal or intravenous administration.

9. A method of treating pain comprising in a subject in need thereof comprising administering to the subject a pharmaceutical composition comprising 600-1200 mg of acetaminophen and 100-10,000 mg of N-acetylcysteine amide (NACA).

10. The method of claim 9, wherein the pharmaceutical composition comprises 100-1000 mg of NACA.

11. The method of claim 9, wherein the pharmaceutical composition comprises 100-500 mg of NACA.

12. The method of claim 9, wherein the pharmaceutical composition comprises 150-300 mg of NACA.

13. The method of claim 9, wherein the pharmaceutical composition further comprises a pharmaceutically acceptable salt or excipient.

14. The method of claim 9, wherein the pharmaceutical composition is a dosage form appropriate for oral administration.

15. The method of claim 14, wherein the dosage form is selected from the group consisting of powders, granules, suspensions, slurries, solutions in water or non-aqueous media, sachets, capsules, gelcaps, lozenges, pills, dragees, gels, syrups and tablets.

16. The method of claim 9, wherein the pharmaceutical composition is a dosage form appropriate for intraperitoneal or intravenous administration.

17. A method of treating N-acetyl-p-aminophenol (APAP) toxicity in a subject in need thereof comprising administering to the subject a therapeutically effective amount of NACA.

18. The method of claim 17, wherein the therapeutically effective amount of NACA is between 100 and 10,000 mg of NACA.

19. The method of claim 18, wherein the therapeutically effective amount of NACA is between 5000 and 10,000 mg of NACA.

20. The method of claim 19, wherein the therapeutically effective amount of NACA is between 7000 and 10,000 mg of NACA.

21. The method of claim 17, wherein the therapeutically effective amount of NACA is between 50 and 200 mg/kg of NACA.

22. The method of claim 17, wherein the therapeutically effective amount of NACA is between 80 and 150 mg/kg of NACA.

23. The method of claim 17, wherein the NACA is administered orally.

24. The method of claim 23, wherein the oral NACA is administered in a dosage form selected from the group consisting of powders, granules, suspensions, solutions in water or non-aqueous media, sachets, capsules, gelcaps, lozenges and tablets.

25. The method of claim 17, wherein the NACA is administered intraperitoneally or intravenously.

26. The method of claim 17, wherein the subject is a mammal.

27. The method of claim 26, wherein the mammal is a human.
